(19) United States
(12) Patent Application Publication
(10) Pub. No.: US 2017/0173551 A1
(43) Pub. Date: Jun. 22, 2017

(54) Device For Evaluation Of At Least One Performance Criterion Of Heterogeneous Catalysts

(71) Applicants: CENTRE NATIONAL DE LA RECHERCHE SCIENTIFIQUE, PARIS (FR); ECOLE CENTRALE DE LILLE, VILLENEUVE D'ASCQ cedex (FR); UNIVERSITE DES SCIENCES ET TECHNOLOGIES DE LILLE - LILLE 1, VILLENEUVE D'ASCQ cedex (FR)

(72) Inventors: Franck DUMEIGNIL, VILLENEUVE D'ASCQ (FR); Sabastien PAUL, THUN SAINT AMAND (FR); Louise DUHAMEL, VILLENEUVE D'ASCQ (FR); Jeremy FAYE, LA MADELEINE (FR); Pierre MIQUEL, BRUXELLES (BE); MIckael CAPRON, BACHY (FR); Jean-Luc DUBOIS, MILLERY (FR)

(21) Appl. No.: 15/117,088

(22) PCT Filed: Feb. 4, 2015
(86) PCT No.: PCT/FR2015/050263

§ 371 Date: Aug. 5, 2016

(30) Foreign Application Priority Data
Feb. 6, 2014 (FR) 1450923

(51) Int. Cl. B01J 19/00; G01N 30/16; C07C 41/09; G01N 30/06; G01N 31/10; B01J 21/04

(52) U.S. Cl. CPC B01J 19/0046; G01N 31/10; B01J 21/04; C07C 41/09; G01N 30/06; G01N 30/16; B01J 2219/00286; B01J 2219/00337; B01J 2219/00704; B01J 2219/00747; G01N 2030/067; G01N 2030/025

(57) ABSTRACT

The invention relates to a device (1) for evaluation of at least one performance criterion of heterogeneous catalysts, comprising; at least one reactant source (2), at least one reaction zone equipped with at least one catalyst and connected to at least one reactant source (2) in such a way as to produce, in each reaction zone, a heterogeneous catalytic reaction between each catalyst present in the reaction zone and the reactant or reactants coming from each reactant source (2) connected to the reaction zone, and means for evaluation of at least one performance criterion of heterogeneous catalysts, characterized in that the device (1) further comprises a gas chromatograph and in that each reaction zone (9) is situated in an injector (8) of the gas chromatograph.

A subject-matter of the present invention is a high output, compact and economical catalytic screening device intended to evaluate the performances of heterogeneous catalysts.

Heterogeneous catalysis represents the branch of catalysis in which the catalyst occurs in another phase, within the thermodynamic meaning of the term, than the reactants and products of the catalysed reaction. The catalyst is typically in the solid state and catalyses a reaction in which the reactants and products are in the liquid phase or in the gas phase.

It is useful to evaluate the performance of a catalyst. In order to do this, there exist several performance criteria which can be assessed. For example, it is possible to determine the degree of conversion of a reactant, that is to say the number of moles of reactants which have reacted divided by the number of moles of this reactant initially present in the reactor (case of a closed reactor), or the ratio of the molar flow rate of this reactant which has reacted to the molar flow rate of this reactant entering the reactor (case of an open reactor), or alternatively the yield of reaction products.

Devices for evaluating the performance of heterogeneous catalysts are known in particular from the document WO 02/092219. However, these devices are sophisticated, bulky and particularly expensive.

The present invention is targeted at overcoming these disadvantages.

It provides in particular a device for evaluating at least one performance criterion of heterogeneous catalysts, comprising:
at least one reactant source,
at least one reaction region provided with at least one catalyst and connected to at least one reactant source, so as to carry out, in each reaction region, a heterogeneous catalysis reaction between each catalyst present in the reaction region and the reactant or reactants resulting from each reactant source connected to the reaction region, and
means for evaluating at least one performance criterion of heterogeneous catalysts.

The device according to the invention additionally comprises a gas chromatograph and each reaction region is located in an injector of the chromatograph.

Thus, instead of using a laboratory reactor normally employed for the measurements of catalytic performance, the device according to the invention is based on the rerouting of the use of an injector of a gas chromatograph in order to use it as fixed bed catalytic reactor. It is thus possible to simultaneously use several injectors in parallel, which makes it possible to accelerate the catalytic tests by adopting a high output catalytic screening methodology. In addition, the device exhibits the advantage of complete reversibility of use if the user no longer desires to use it as catalytic screener but again in its original function of chromatograph.

The device preferably comprises at least two reaction regions.

Each catalyst can be in the solid state and each reactant can be in the gas state.

The performance criterion can be chosen from the degree of conversion of a reactant and the yield of reaction products.

Each reaction region is advantageously a liner of the injector.

Each liner can comprise a catalytic bed.

The device can comprise several injectors positioned in parallel.

The device can comprise a module capable of independently regulating the temperature and the pressure of each reaction region (i.e., each injector) and the feed flow rates of each reaction region.

The gas chromatograph can additionally comprise a sampling system, at least one chromatographic column and at least one detection system, in particular a detection system conventionally used in gas chromatographs, for example a flame ionization detector (FID), a thermal conductivity detector (TCD) or any other detector known to a person skilled in the art.

The device can comprise a source of carrier gas, the carrier gas being intended to dilute and transport the reactant or reactants in each reaction region.

The reactant or reactants and the carrier gas entering each reaction region can result from an evaporator, followed by a system for distributing the streams.

Another subject-matter of the invention is a process for evaluation of at least one performance criterion of heterogeneous catalysts, employing the device described above.

The process according to the invention comprises:
in each reaction region, a stage of heterogeneous catalysis reaction between the catalyst(s) present in the reaction region and the reactant(s) resulting from each reactant source connected to the reaction region, and
a stage of evaluating at least one performance criterion of each catalyst, in particular in each reaction region.

Another subject-matter of the invention is the use of a device described above as a catalytic screener or as a chromatograph, it being possible to use the device in a reversible manner as a catalytic screener or as a chromatograph.

Other characteristics and advantages of the present invention will become more clearly apparent on reading the following description, given by way of illustrative and nonlimiting example and made with reference to the appended drawings, in which:

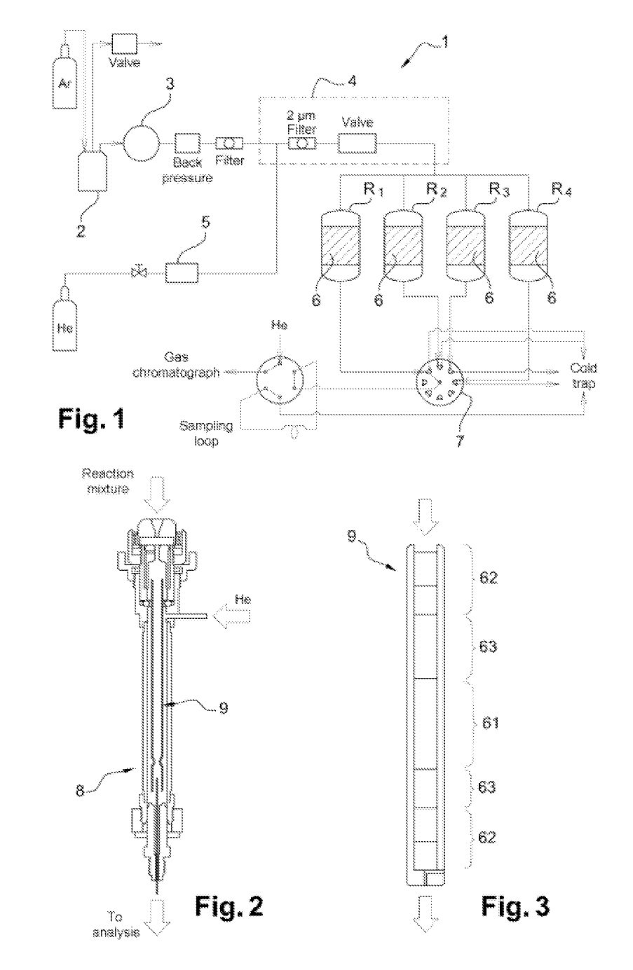

FIG. 1 diagrammatically illustrates a device for the evaluation of the performance of a heterogeneous catalyst according to the invention,

FIG. 2 is a view in longitudinal cross section of an injector used in the device according to the invention, and

FIG. 3 is a view in longitudinal cross section of a liner of the injector.

FIG. 1 illustrates a catalytic screening system 1 applied, by way of example, to the conversion of methanol. A tank 2 with a capacity of one litre is filled to three quarters of its capacity with 99.99% methanol. This tank 2 is slightly pressurized under argon using an Ar cylinder in order to degas the reactant (that is to say, to discharge the oxygen and the nitrogen dissolved at ambient temperature and atmospheric pressure) and also to promote the feeding of a pump 3.

The liquid methanol is introduced into an evaporator 4 according to a predetermined flow rate (for example 100 μl.min⁻¹), the evaporator 4 being simultaneously fed with carrier gas (for example helium, using an He cylinder, at a flow rate of 160 ml.min⁻¹) by means of a weight flow controller 5. The two compounds (gaseous helium and liquid methanol) enter the evaporator 4, which comprises a tube filled with silicon carbide, the diameter of the particles of which is, for example, 125 μm, and which is maintained at 120° C. The objective is, at this point of the device 1, to vaporize the methanol and to ensure that the gaseous reaction mixture to be distributed over the different reactors is homogeneous. By way of example, four reactors R₁, R₂, R₃ and R₄ have been represented in FIG. 1.

The outlet of the evaporator 4 is connected to a stream distributor equipped with an inlet and four outlets, to which four tubes of fused silica, with an internal diameter equal to 0.1 mm and of a length of 40 cm, are connected, in order to generate an individual pressure drop thirty times greater than that produced by the catalytic bed (2.25×10³ Pa per reactor R₁, R₂, R₃ and R₄). The pressure drop generated by this stream distributor is from approximately 9 to 10 bar.

The reaction mixture thus results from a module. The module, which will be linked immediately above the reactors R₁, R₂, R₃ and R₄, makes it possible to prepare the feed mixture which will be injected into the reactors R₁, R₂, R₃ and R₄ and to distribute it equitably between the different routes. It is possible to prepare therein a gaseous reactant mixture but also to vaporize liquids before mixing with gases and introduction into the reactors. The module is thus a kind of oven comprising evaporators, tubes, mixing regions, devices for controlling and regulating the flow rates, and heating elements, such as electrical resistances. The configuration of this module can be adjusted as a function of the reactions to be studied.

The module makes it possible in particular to regulate the temperature of each reactor R₁, R₂, R₃ and R₄ and the feed flow rates. It is also possible to control the pressure of the reaction by adding a blow-off valve at the reactor outlet. Software can ensure the control of the module, for example with inputs of temperature setpoints or of flow rates, or also the indication of true values. The regulation of the temperature can be an external control or a control provided by the chromatograph itself.

In accordance with the invention, the reactors R₁, R₂, R₃ and R₄ of the device 1 for evaluation of the performance of the catalyst are injectors of a gas chromatograph. The remainder of the chromatograph may or may not be used.

A gas chromatograph typically comprises:
an oven, which makes possible adjustable temperature programming and which can also be equipped with a rapid cooling system;
an injection system, which will make it possible to introduce and to render volatile the sample to be analyzed. The injection can be carried out manually or automatically using a sampler;
a column, on which the different molecules of the injected sample will be separated according to their affinities with the stationary phase of the said column;
a detection system, which will make it possible to measure the signal emitted by the different molecules, to be able to identify them and to quantify them after calibration;
a pressure reducing/regulating system for the gases used (helium, hydrogen, nitrogen and compressed air). Modern chromatographs contain electronic systems for regulating the gases, which are also purified by filter cartridges.

The operating principle of the chromatograph is as follows. The sample (a volatile liquid or a gas) is first introduced into the injector placed at the column top via a sampler or a microsyringe which will pass through a rubber disc, known as septum, in order to be reencountered in a small chamber upstream of the column, known as insert. The carrier gas passes through the injector and the latter is brought to a temperature appropriate to the volatility of the sample.

Subsequently, once rendered volatile, the different compounds of the sample will be swept along by the carrier gas through the column and be separated from one another as a function of their affinity with the stationary phase. The stationary phase can be a non-volatile or only very slightly volatile liquid (gas-liquid chromatography) or an absorbent solid (gas-solid chromatography). In both cases, the stationary phase will bring about a phenomenon of chromatographic retention with the different compounds, known as solutes. The greater the affinity of the compound with the stationary phase, the more time it will take to exit from the column. The raw experimental quantity is known as retention time. This is the time which elapses between the injection of the sample and the appearance of the maximum signal of the solute at the detector. In order to promote the transportation of all the compounds through the column (elution), it is necessary to determine the satisfactory temperature of the oven. In general, the temperature should be slightly greater than the boiling point of the compounds, so that the compounds do not exit too soon, which would have the consequence of having their peaks mixed up with that of the dead time. It is possible to operate under isothermal conditions, that is to say with a fixed temperature throughout the analysis, or with a temperature programme which varies.

At the outlet of the column, the compounds encounter an essential component which is referred to as detector. This component continuously evaluates the amount of each of the constituents separated within the carrier gas by virtue of the measurement of different physical properties of the gas mixture. The detector sends an electronic signal to a recorder, which will draw the curves of each peak as a function of their intensities (curve of Gaussian type). The set of peaks is referred to as chromatogram.

A gas chromatograph can comprise several analytical routes, in particular from two to four, which are injector+column+detector combinations.

The injectors are housed in a metal block, the temperature of which is regulated in order to ensure good thermal homogeneity of the system. The sample will be vaporized and the solutes will pass through the injector through a glass (sometimes metal) tube, known as a liner, by virtue of the carrier gas, as far as the top of the column. The advantage of the liner is to retain the non-volatile constituents of the sample, unsuitable by nature to chromatography.

In the case of the use of the injector for the determination of catalytic performance, each liner is filled with the solid catalysts to be tested, in the powder form, and is traversed continuously by a gaseous reaction mixture.

As illustrated in FIG. 1, the reaction mixture is subsequently simultaneously directed towards the four reactors R₁, R₂, R₃ and R₄ and is thus brought into contact with a catalytic bed 6 at a given temperature. The stream at the output of each reactor R₁, R₂, R₃ and R₄ is, via a multiposition valve 7, either analysed via an injection loop connected to an analytical system external to the device described here or collected using a cold trap of sparger type, in order to recover the desired products. The performance of the catalyst can be evaluated using another gas chromatograph or using the same gas chromatograph as that receiving the catalytic screening system.

In accordance with the invention, the conventional use of a gas chromatograph is rerouted by using the liner 9 of the injector 8 as catalytic reactor Rᵢ, (FIG. 2).

This operation is carried out by replacing the content of the liner 9, normally mineral wool, with the catalytic bed 6 comprising the catalyst 61, as well as, for example, quartz wool 62 and silicon carbide 63, as illustrated in FIG. 3. By way of example, the liner 9 thus is completely emptied in order to be successively filled with:
quartz wool 62, in order to ensure the stability of the catalytic bed,
silicon carbide 63 (125 μm),
the catalyst 61 (200 mg) mixed with silicon carbide (200 mg),
silicon carbide 63 (125 μm),
quartz wool 62.

The present invention is described in more detail by the following example, to which, however, it is not limited.

### EXAMPLE

### Evaluation of the Catalytic Performance of an Alumina for the Dehydration of Methanol

The dehydration of methanol to give dimethyl ether is carried out according to the following reaction:

CH₃OH→CH₃OCH₃+H₂O

Experimental Conditions

The catalyst tested is a gamma alumina γ-Al₂O₃ sold by Alfa Aesar.

The carrier gas is a mixture comprising 1 mol % of Kr in He, into which the methanol is evaporated in order to obtain the following molar proportions for the reaction mixture: MeOH/He/Kr: 27.4/71.9/0.7 (mol %).

The sum of the gas flow rates is 33 000 ml.h⁻¹.g⁻¹ at 25° C. and atmospheric pressure.

Stability of the Composition of the Reaction Mixture

Before each reaction, a series of six analyses per reactor is carried out in order to confirm the stability of the composition of the reaction mixture. The number of moles of methanol shown for each reactor in Table 1 is a mean of the six analyses:

TABLE 1

| | R₁ | R₂ | R₃ | R₄ |
|---|---|---|---|---|
| n_{MeOH} | 668.9 | 677.7 | 666.9 | 667.9 |
| (10⁻⁹ mol) | | | | |
| Standard deviation | 16.4 | 20.2 | 16.4 | 15.9 |
| (10⁻⁹ mol) | | | | |
| Relative standard | 2.5 | 3.0 | 2.5 | 2.4 |
| deviation (%) | | | | |

Good stability is observed for all the reactors.

Conversion of the Methanol

The degree of conversion of the methanol, and also the relative standard deviation between the reactors for six analyses per reactor, is shown in Table 2:

TABLE 2

| | | | | | | Relative standard |
|---|---|---|---|---|---|---|
| | R₁ | R₂ | R₃ | R₄ | Mean | deviation (%) |
| Conversion | 83.4 | 84.0 | 82.6 | 83.0 | 83.3 | 0.7 |
| (%) | | | | | | |

It is found that the reproducibility of the degree of conversion of the methanol from one reactor to the other is excellent.

### Yield of Dimethyl Ether

The yield of dimethyl ether for each reactor is shown in Table 3:

TABLE 3

| | | | | | | Relative standard |
|---|---|---|---|---|---|---|
| | R₁ | R₂ | R₃ | R₄ | Mean | deviation (%) |
| Yield of | 87.0 | 83.4 | 85.5 | 85.7 | 85.4 | 1.7 |
| dimethyl | | | | | | |
| ether (%) | | | | | | |

Good reproducibility of the catalytic performance is observed between the different reactors.

The invention thus makes it possible to obtain an economical, compact and simple device for measuring the performance of heterogeneous catalysts, in particular in the gas phase. The different injectors of the chromatograph, rerouted from their original application in order to become reactors, can be used simultaneously in parallel, which makes it possible to greatly accelerate the catalytic tests using the methodology of high output catalytic screening. The technology used in addition exhibits the advantage of complete reversibility of use if it is no longer desired to use it as catalytic screener but again in its original function of chromatograph. It is possible in addition to simultaneously test several catalysts, or just one catalyst under different operating conditions (temperature or contact time, for example). The time necessary in order to carry out the measurements of the catalytic performance of the catalysts is thus significantly reduced and the time for the development of a new heterogeneous catalyst is thus decreased.

1. Device for evaluating at least one performance criterion of heterogeneous catalysts, comprising:
at least one reactant source,
at least one reaction region provided with at least one catalyst and connected to at least one reactant source, so as to carry out, in each reaction region, a heterogeneous catalysis reaction between each catalyst present in the reaction region and the reactant or reactants resulting from each reactant source connected to the reaction region, and
means for evaluating at least one performance criterion of heterogeneous catalysts, characterized in that the device additionally comprises a gas chromatograph and in that each reaction region is located in an injector of the gas chromatograph.

2. Device according to claim 1, further comprising at least two reaction regions.

3. Device according to claim 1, wherein each catalyst is in the solid state and in that each reactant is in the gas state.

4. Device according to claim 1, wherein the performance criterion is chosen from the degree of conversion of a reactant and the yield of reaction products.

5. Device according to claim 1, wherein each reaction region is a liner of the injector.

6. Device according to claim 5, wherein each liner comprises a catalytic bed.

7. Device according to claim 1, further comprising several injectors positioned in parallel.

8. Device according to claim 1, further comprising a module capable of independently regulating the temperature and the pressure of each reaction region and the feed flow rates of each reaction region.

9. Device according to claim 1, further comprising a sampling system, at least one chromatographic column and at least one detection system.

10. Device according to claim 1, further comprising a source of carrier gas, the carrier gas being intended to dilute and transport the reactant or reactants in each reaction region.

11. Device according to claim 10, wherein the reactant or reactants and the carrier gas entering each reaction region result from an evaporator, followed by a system for distributing the streams.

12. Process for evaluation of at least one performance criterion of heterogeneous catalysts, employing a device according to claim 1, said method comprising the step of:
in each reaction region, a stage of heterogeneous catalysis reaction between the catalyst(s) present in the reaction region and the reactant(s) resulting from each reactant source connected to the reaction region, and
a stage of evaluating at least one performance criterion of each catalyst.

13. A catalytic screener or a chromatograph, comprising:
Said catalytic screener or said chromatograph comprising a device for evaluating at least one performance criterion of heterogeneous catalysts according to claim 1.
